(19) United States
(12) Patent Application Publication
(10) Pub. No.: US 2002/0133144 A1
(43) Pub. Date: Sep. 19, 2002

(54) Laser irradiation mapping system

(75) Inventors: Kin Foong Chan, Plano, TX (US); Jeffrey Whitman, Dallas, TX (US); Suzanne I. Ahn, Dallas, TX (US)

Correspondence Address: HOWISON, THOMA & ARNOTT, L.L.P, P.O. BOX 741715, DALLAS, TX, 75374-1715, US

(73) Assignee: Ball Semiconductor, Inc.

(21) Appl. No.: 09/812,219

(22) Filed: Mar. 19, 2001

(51) Int. Cl. A61B 18/20

(52) U.S. Cl. 606/4

(57) ABSTRACT

A digital micromirror device is used in a semi-automated system to reflect a tailored portion of a laser beam according to image data that defines a mapped target area of an object for selective irradiation by the tailored laser beam. A computer having an interface device, such as a light pen, is used to map the boundary on a computer screen of a target area of an object, which may be the retina of a patient's eye or some other object. A projector cooperates with a laser source, the digital micromirror device and the computer to enable a laser beam to be tailored to correspond to the irregular shape of the intended target area to avoid irradiating portions of the object other than the target area itself. The system is capable of tracking movement of the target area and maintaining the tailored laser beam aligned to irradiate the target area during movement. The individual micromirrors of the digital micromirror device can be time-division modulated to vary the dose of irradiation across the target area according to a gray-scale map of the image of the target area.

### TECHNICAL FIELD OF THE INVENTION

The present invention generally relates to laser treatment systems, and more particularly to systems and methods for precisely targeting an object for irradiation by a laser beam.

### BACKGROUND OF THE INVENTION

A laser beam is a coherent beam of monochromatic light. It is ideally suited for various surgical procedures due to its ability to concentrate high energy at a small tissue site. A most suitable application is in eye surgery, though other organs of the body can be, and increasingly are being, treated with lasers.

The popularity of refractive surgery to correct common visual impairments such as myopia has contributed to the development of high-quality laser treatment equipment, such as excimer laser equipment made by a number of manufacturers. [The term “excimer” is a contraction of the expression “excited dimer,” which describes the photochemical process that emits light at a wavelength of 193 nanometers.] U.S. Pat. No. 6,139,542, entitled “Distributed Excimer Laser Surgery System,” describes one such refractive surgery system.

Other uses of lasers in ophthalmic surgery are known, and increasingly sophisticated types of laser treatment equipment are being made available to practitioners. For example, U.S. Pat. No. 6,066,128, entitled “Multi-Spot Laser Surgery,” describes use of a laser in a panretinal photocoagulation procedure. This particular patent discloses a technique for splitting a single beam of laser energy into a plurality of parallel laser beams for more efficient treatment.

The problem of accurately directing a laser beam at particular tissue has been addressed in the prior art. For example, U.S. Pat. No. 4,520,824, entitled “Instrument for Ophthalmic Laser Surgery,” describes an apparatus that permits the physician to move a laser beam in two horizontal directions using an “X-Y table” to accurately align the beam with a treatment spot in a patient's eye. This patent also discloses a beam-directing system of mirrors that permits coaxial alignment of a low-powered aiming laser with a high-powered therapeutic laser.

Problems associated with eye movement during ophthalmic laser surgery also have been addressed in the prior art. For example, U.S. Pat. No. 5,865,832, entitled “System for Detecting, Measuring and Compensating for Lateral Movements of a Target,” describes a system in which a servo-controlled pivoting mirror responds to eye movements in real time so that a treating laser beam tracks the intended target. A similar tracking system is disclosed in U.S. Pat. No. 6,099,522, entitled “Automated Laser Workstation for High Precision Surgical and Industrial Interventions,” which also discloses a technique for selecting a pattern of laser treatment points according to the shape of the target tissue.

### SUMMARY OF THE INVENTION

When an area of an object to be treated with a laser beam is irregular in shape, it becomes time consuming to apply multiple small diameter laser beams to the treatment area. When the treatment area involves vital tissue, such as the retina, it is undesirable to use a wide-area beam that covers more than the irregularly-shaped treatment area, possibly damaging or destroying adjacent healthy tissue. The problem of accurately and efficiently treating an irregularly-shaped area of tissue with a laser without significantly impairing adjacent healthy tissue has not been adequately addressed in the art of laser surgery.

The present invention provides an apparatus or assembly of semi-automated equipment that enables a treating physician to map a target area of tissue for exposure by a laser beam that is tailored in shape to accurately conform to the irregular boundaries of the target area selected by the physician. The equipment includes as fundamental components: a laser source, a projector/imager, an array of selectively positionable micromirrors, and a computer system. In operation, the computer system converts an image of an area of tissue, such as the retina of a patient's eye, into a digitally stored image. The physician maps a target area of the tissue for treatment by a correspondingly-shaped laser beam, which can be accomplished in various ways. For example, a light pen can be used to draw a boundary around the target area on a computer screen that is displaying an image of the retina or other tissue to be treated. Other mapping techniques are within the scope of the invention, as are nonsurgical applications. Once the target area is mapped, corresponding micromirrors within a two-dimensional array are selected by the computer system to move to their ON positions. Then, a laser beam generated by the laser source is directed at the array including both the ON-position and OFF-position micromirrors. A portion of the laser beam is thus reflected by the ON-position micromirrors and redirected by the projector/imager at the target area of the object under treatment so that only the target area is irradiated.

### BRIEF DESCRIPTION OF THE DRAWINGS

For a more complete understanding of the present invention and its advantages, the following detailed description is provided in conjunction with the accompanying drawings, in which:

FIG. 1 is a block diagram illustrating the assembled major components of a preferred embodiment of the present invention;

FIG. 2 is a schematic diagram illustrating a possible use of the invention in an ophthalmic surgical procedure;

FIG. 3 is a schematic cross-sectional view illustrating devices within one of the major components of the preferred embodiment of FIG. 1, specifically FIG. 3 shows the internal devices of a DMD module;

FIG. 4 is a greatly enlarged schematic plan view of a portion of a digital micromirror device, which is one of the internal devices of the DMD module of FIG. 3;

FIG. 5 is a schematic cross-sectional view (some cross-hatching being left out for clarity) of the device of FIG. 4 taken along line V-V in FIG. 4;

FIG. 6 is a schematic view similar to FIG. 5 but with elements of the device assuming different positions; and

FIG. 7 is a schematic cross-sectional view illustrating devices within another major component of the embodiment of FIG. 1, specifically FIG. 7 shows mirrors within a mirror chamber of a projector.

### DESCRIPTION OF THE PREFERRED EMBODIMENTS

Referring to FIG. 1, a preferred embodiment of a particular application of the invention is illustrated in which an apparatus comprising an assembly of semi-automated equipment is designated generally by reference numeral 10. The apparatus 10 is shown in block-diagram form, one of its major components being a projector, which is generally designated by reference numeral 12, wherein the particular application depicted is a laser surgical procedure for treating a patient's eye 14.

Other applications of the invention and corresponding adaptations of the illustrated apparatus 10 will suggest themselves to those skilled in the art upon reading this description. Some other applications are specifically mentioned below, but it will be appreciated that the invention has very useful application in the field of ophthalmic laser surgery. By way of example, the invention will be described in the context of an application for the treatment of the common eye condition known as age-related macular degeneration or AMD. Such treatment involves irradiating a particular spot or target area on the retina of patient's eye, and perhaps repeating the procedure on other target areas of the retina. This is one type of treatment in the general field of photodynamic therapy or PDT in which the invention has useful application.

The projector 12 has a frame 16 that supports the projector's component parts between a viewing end 18 and an imaging end 20. The viewing end 18 preferably consists of a conventional binocular viewer, but any suitable lens system will suffice. The major component parts of the projector 12 define a high-precision optical system preferably comprising the binocular viewer 18, a centrally-located mirror chamber 22, a fundus camera 24 located between the mirror chamber and the imaging end 20, and a filter box 26 located between the mirror chamber and the binocular viewer 18.

The filter box 26 preferably includes a dichroic filter (not shown) for protecting the physician's eyes from laser light. It will be appreciated that a pair of dichroic mirrors may perform the filter function to prevent light at selected wavelengths from reaching the physician's eyes. Projector controls 28 are mounted on the frame 16 for positioning the imaging end 20 relative to the patient's eye 14, and for controlling the projection of a tailored laser beam 30 at a target area of the ocular findus in the patient's eye. The fundus camera 24 includes a light source 32, an example of which may be a 12 volt, 50 watt halogen lamp commonly used in conventional fundus camera equipment.

It will be appreciated that the fundus camera 24 can be replaced by another type of lens system, such as an ophthalmoscope, or a customized lens system that performs the necessary projecting and imaging functions. Lens systems for fundus cameras and ophthalmoscopes are well known in the art. For example, see U.S. Pat. No. 5,572,266.

The apparatus 10 further comprises a laser source 34, which controllably emits a primary laser beam 36, and a DMD module 38, which receives the primary laser beam 36 and selectively emits a secondary laser beam 40 consisting of a portion of the primary laser beam 36. The secondary laser beam 40 is reflected by the mirror chamber 22 and projected by the findus camera 24 as laserbeam 30, which is tailored to conform to the specific shape of the target area in the patient's eye. The apparatus 10 also comprises a computer system, which in the preferred embodiment of the invention includes a digital camera 42, a computer 44 and a DMD controller 46.

An image of the ocular fundus of the patient's eye 14 is created by illuminating the ocular fundus using the light source 32 in the fundus camera 24. The reflected illuminated image is seen by the physician using the binocular viewer 18, and is redirected by the mirror chamber 22 toward the digital camera 42. The redirected illuminated image is designated by reference numeral 48 in FIG. 1.

The digital camera 42 may be any conventional CCD camera, which is a well-known type of digital imaging equipment. An example of a CCD camera suitable for use with a fundus camera is disclosed in U.S. Pat. No. 5,140,352. Other types of digital imaging equipment can be substituted for the CCD camera 42 in the apparatus of the invention. The digital imager or CCD camera 42 creates a digitally stored image of the reflected image 48, and transmits digital data representative of the reflected image 48 to the computer 44 through a computer input bus 50.

The computer 44 includes the input bus 50, an output bus 52, a screen 54 for displaying images, a main processor 56, and a keyboard 58, all of which may be conventional components of a general-purpose personal computer capable of running standard software. Ordinarily, the computer 44 also will include a conventional disk drive for storing permanent records and downloading software.

In the preferred embodiment of the invention illustrated in FIG. 1, the computer 44 also includes an interface device that is specially adapted for defining a target area of a visual image on the screen 54. The interface device preferably comprises a conventional light pen 60 that is operatively connected to the main processor 56 through a flexible cable 62. Such light pens and systems for interacting with computer screens are well known in the art. As an example, one such light pen system is described in U.S. Pat. No. 5,187,467. As an alternative, a conventional computer mouse can be used to move a curser on the screen 54 to define the target area. As a further alternative, a programmed system for semi-automatically defining the target area can be employed, an example of which is described below following the description of the preferred mirror chamber 22 of FIG. 7.

Briefly, the operation of the apparatus 10 to selectively irradiate a target area of a patient's retina is as follows. The projector 12 illuminates the patient's eye 14 using the light source 32 of the fundus camera 24. A reflected image of the retina is redirected by the mirror chamber 22 as illuminated image 48. The CCD camera 42 creates a digitally stored image of the illuminated image 48 and transmits data representative of the image to the computer 44 through bus 50. The computer 44 displays a visual image on the screen 54 corresponding to the illuminated image 48. The physician or an assistant operator uses the light pen 60 to manually draw a boundary around a target area 64 within the image on the screen 54, thereby digitally mapping the target area 64 within the computer 44. During this procedure, the physician can directly view the reflected image of the patient's eye through the binocular viewer 18.

The computer 44 transmits data corresponding to the mapped target area 64 over the computer's output bus 52 to the DMD controller 46, where it is stored in a memory 66. The DMD controller, operating under the direction of a microprocessor 68, transfers data from the memory 66 and control signals from the microprocessor 68 to the DMD module 38 over a transfer bus 70. The DMD module 38 thus assumes a state that will selectively reflect a portion of the primary laser beam 36 and emit the portion out as the secondary laser beam 40, which will correspond in shape to the mapped target area 64. When the physician is satisfied that the mapped target area is suitable for irradiation, he or she uses the controls 28 on the projector 12 to send a signal over a first signal line 72 to the laser source 34 to trigger emission of the primary laser beam 36, which is partially reflected by the DMD module 38 as the secondary laser beam 40. The projector 12 redirects the secondary laser beam 40 as the tailored laser beam 30 to impinge on the target area of the retina within the patient's eye 14.

It will be appreciated from the foregoing that more than one target area within the image field can be mapped and simultaneously irradiated by separate, parallel laser beams, each corresponding in shape to its respective target area.

As a safety precaution, the laser source 34 will not emit the laser beam 36 unless it is simultaneously receiving a ready signal on a second signal line 74 that is connected to the computer 44. As one of various possible ways of setting up the ready signal, the operator uses the light pen 60 to position a box 76 at an area of the image of the patient's retina on the screen 54 that includes a transition from relatively dark to relatively bright light intensity. For example, the box 76 can be positioned to include the image of a portion of a blood vessel. During intervals in which the computer 44 does not detect movement of the eye out of correct position using a light intensity map within the box 76 as a reference, the ready signal is transmitted on signal line 74. However, whenever the light intensity map within the box 76 changes so that movement beyond a tolerance from the initial set-up position is detected, the computer 44 sends a disable or “not ready” signal on signal line 74. This prevents emission of the primary laser beam 36 by the laser source 34, and of course no resulting beam 30 is emitted to impinge on the retina of the patient's eye 14. Other safety techniques for assuring proper eye position before the laser beam 36 is emitted by the laser source 34 can be employed as alternatives to the above-described technique.

Additionally, an automatic tracking technique can be employed. For example, as a preferred technique, the computer 44 is programmed to track movement of the target area 64 in real time on the screen 54, and continuously send updated data identifying the position of the target area 64 to the memory 66. It is ordinarily necessary to track movement of the target area 64 indirectly while it is being irradiated, since laser light reflected by the retina may prevent direct detection of the boundary of the target area. Therefore, a suitable reference point or small area removed from the target area 64, such as the area within the box 76 mentioned above, is used to calculate corresponding movement of the target area. Two or more such reference points can be used rather than just one, so long as they move in a way that accurately predicts corresponding movement of the target area 64. Any of various suitable automatic tracking techniques can be employed, including techniques similar to those described in U.S. Pat. Nos. 5,865,832 and 6,099,522, which are hereby incorporated by reference.

The updated position data derived by a suitable automatic tracking technique can be captured by the computer 44 and transmitted to the memory 66 at a very fast rate. The microprocessor 68 can then send a complete new memory map including the position of the target area 64 to the DMD module 38 at a refresh rate that exceeds the rate of any significant eye movement and is less than the frequency response of the DMD module 38. For example, the refresh rate may be between 0.1 and 5.0 kHz.

Accordingly, the position of the secondary laser beam 40 within the image field automatically tracks minor eye movements. Likewise the tailored laser beam 30 tracks with such minor eye movements so that treatment can continue uninterrupted perhaps completing the entire irradiation treatment in a few seconds or less. Only if a major eye movement occurs in which the target area 64 moves out of the image field, or suddenly disappears (e.g., the patient blinks), will the computer 44 then send a disable signal on signal line 74 to the laser source 34 to inhibit firing of the primary laser beam 36.

PDT treatment for neovascularization conditions such as AMD is typically done with a relatively low intensity laser, which typically will operate at a wavelength in the range from about 500 to about 1000 nanometers. Infrared diode lasers, helium-neon lasers and other types of relatively low intensity lasers are available for use in PDT treatment and various other medical applications according to the invention. As previously noted, the present invention is not limited to ophthalmic laser surgery and can have many other uses, including other types of tissue treatment as well as industrial applications in which the irradiated object is inanimate. Types of tissue treatment can include, for example, tumor ablation, scar tissue treatment and cosmetic surgery applications. Industrial uses may include semiconductor device fabrication and the manufacture of microelectromechanical systems or MEMS. Other industrial applications may use high intensity lasers, such as a CO2 laser or an erbium:YAG (yttrium-aluminum-garnet) laser, such as in precision tool manufacturing processes. The CO2 laser operates at wavelength of 10.6 microns and the Er:YAG laser operates at 2.94 microns. The various components of the apparatus 10 can be adjusted for the particular application and type of laser used in the system.

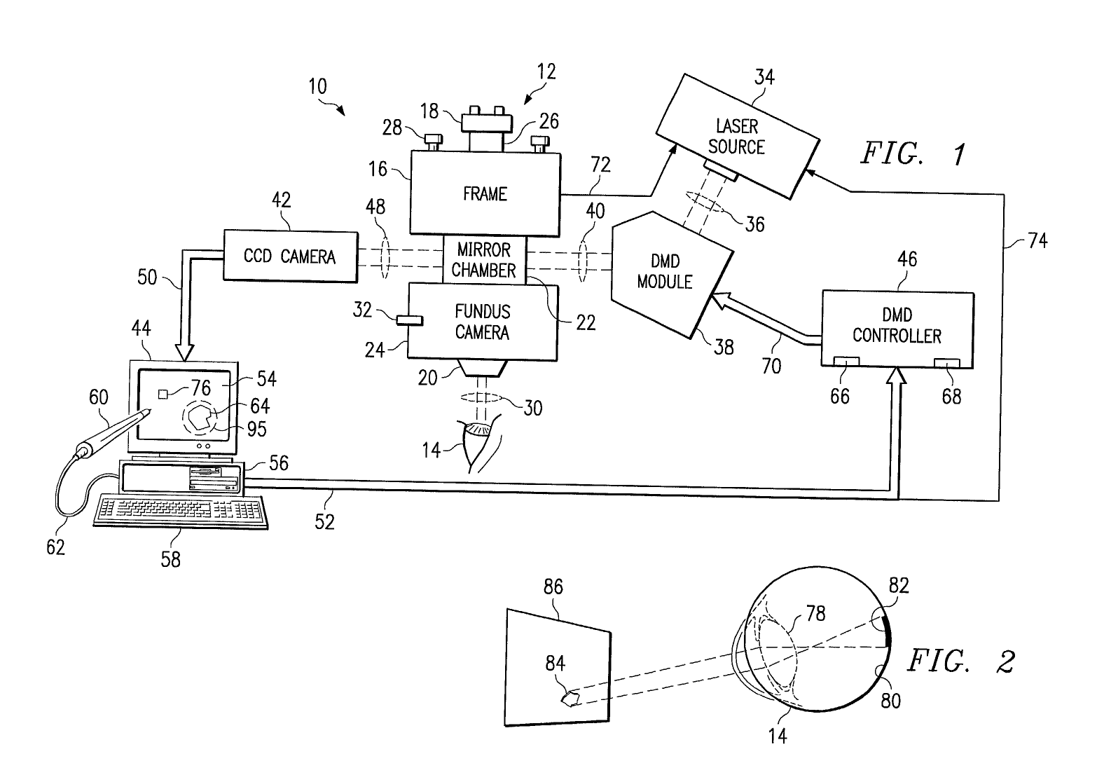

FIG. 2 schematically depicts how the illuminated retina of a patient's eye might reflect an image within the projector 12 of FIG. 1. In FIG. 2, the patient's eye 14 is depicted in a schematic side cross-sectional view. Numeral 78 designates the lens of the eye, and numeral 80 designates the retina. A lesion 82 is identified as the target area of the retina 80 that will be irradiated by a laser beam tailored to conform to the shape of the lesion as determined by the physician using the above-described laser irradiation mapping system. The reflected image of the target area is designated by reference numeral 84, which is a projection onto a hypothetical viewing plane defined by a rectangular viewing area 86 (which appears as a trapezoid in perspective). The actual reflected image 84 of the lesion will be seen by the physician within a circular viewing area of the viewing plane. The circular viewing area surrounding the lesion 82 will include the portions of the retina 80 that are not being treated. As shown in FIG. 1, the reflected image of the retina is also redirected by the mirror chamber 22 of the projector 12 as image 48 seen by the CCD camera 42. The corresponding digitized image including the target area 64 is displayed on the computer screen 54.

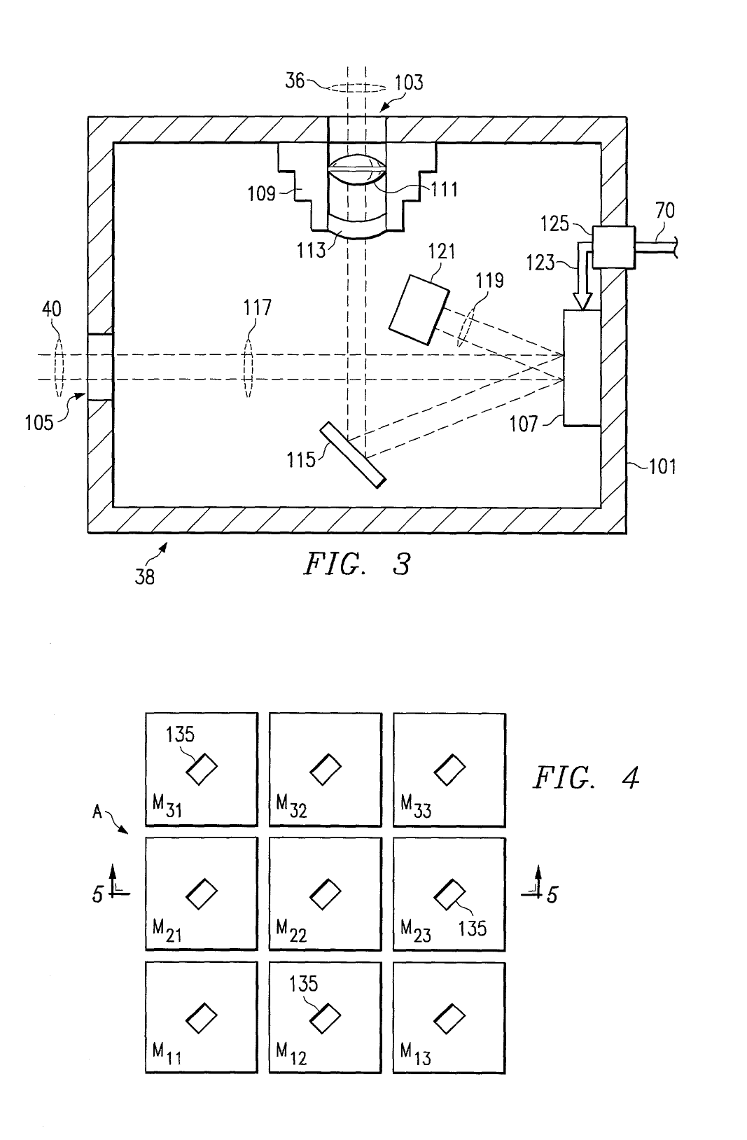

Referring to FIG.3, the internal devices of the preferred DMD module 38 will be described. The DMD module 38 has a housing 101 consisting of peripheral walls shown cross-hatched. The walls of the housing 101 define an entry port 103 and an exit port 105. A digital micromirror device 107 is mounted in the DMD module 38 on a wall of the housing 101 opposite from the exit port 105. An optical instrument 109 is mounted in the housing 101 at the entry port 103. The optical instrument 109 receives the primary laser beam 36 from the laser source 34 (see FIG. 1) through the entry port 103. The optical instrument 109 includes lenses 111 and 113 for directing the laser beam 36 at a mirror 115 that reflects the laser beam 36 at the digital micromirror device 107.

The digital micromirror device 107 is controllable to selectively reflect increments of the primary laser beam 36 along a first path 117 and reflect the remaining portions of the beam 36 along a second path 119. Laser light reflected along the first path 117 passes out of the DMD module 38 through the exit port 105 as the secondary laser beam 40 (see FIG. 1). Laser light reflected along the second path 119 impinges on and is absorbed by an absorber 121, which maybe any suitable beam dump commonly used with laser equipment. The preferred digital micromirror device 107, which is described in greater detail below, is controlled by signals on a bus 123 that is connected to bus 70 through connector 125 affixed in a wall of the housing 101. The bus 70 is the previously described transfer bus 70 that provides data and control signals from the DMD controller 46 (see FIG. 1).

It should be apparent that the schematic nature of FIG. 3 only indicates the general orientation of the beams of laser light that pass through the DMD module 38, their actual sizes and shapes differing. In particular, as the primary beam 36 passes through lenses 111 and 113, its diameter may change. The diameter of the beam is preferably slightly larger than the mirrored surface of the digital micromirror device 107 so that the entire mirrored surface, which typically will be rectangular, can be utilized efficiently in the system. Only a small portion of the beam is reflected by the digital micromirror device 107 along path 117, the remaining portions being reflected along path 119. Thus, in the case of the automatic tracking technique described above, movement of the mapped target area within the image field corresponding to the mirrored surface of the digital micromirror device 107 does not prevent continuous treatment using the automatic tracking technique.

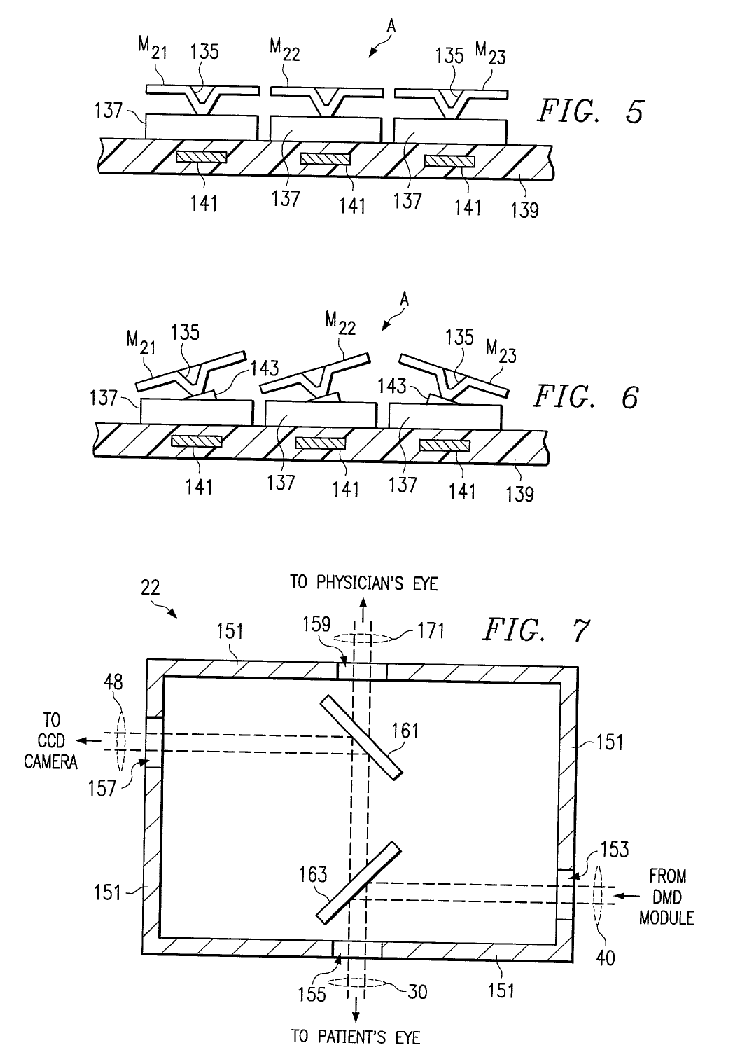

Now referring to FIGS. 4 and 5, the basic structure of the digital micromirror device 107 of FIG. 3 will be described. FIG. 4 shows nine micromirrors M in a 3×3 subarray A bearing the designators M₁₁ through M₃₃ wherein the subscript numbers designate the row and column numbers of each individual micromirror M. The illustrated subarray A is a small portion of a much larger array. For example, the entire digital micromirror device preferably may consist of over 500,000 micromirrors arranged in a 848×600 two-dimensional array. The individual micromirrors M each preferably measure 16 microns on a side with a one micron space between the edges of adjacent micromirrors. Thus, the entire two-dimensional array will present a rectangular image field of about 14 by 10 millimeters. Each micromirror M is supported from below by a centrally-located support post, several of which are designated by reference numeral 135. As seen in FIG. 5, the support posts 135 are V-shaped extensions of the flat plates that define the reflective surfaces of the micromirrors M. The structural details and method of manufacturing such a digital micromirror device are described in U.S. Pat. No. 5,583,688, the disclosure of which is hereby incorporated by reference.

With specific reference to FIG. 5, micromirrors M₂₁, M₂₂, and M₂₃ are shown in a schematic cross-sectional view. Each micromirror M is supported by a base 137, and each base 137 is supported by a common substrate 139. The substrate 139 is a semiconductor substrate (shown greatly simplified) that includes addressable cells 141 therein, each micromirror M having a corresponding cell 141 located beneath it. It will be appreciated from reading U.S. Pat. No. 5,583,688 that the structure of each micromirror M is more complicated than is described herein, the structural details not being necessary to a complete understanding of the present invention, which is mainly system oriented.

Now referring to FIG. 6, the micromirrors M₂₁, M₂₂, and M₂₃, are shown rotated form their rest positions under the influence of an electrostatic drive system that is described in U.S. Pat. No. 5,583,688. Micromirrors M₂₁ and M₂₂ are shown rotated to the left and micromirror M₂₃ is shown rotated to the right. Each post 135 of each micromirror M is attached to a yoke 143 that permits independent rotation of each micromirror. The yokes 143 are only partially visible in FIG. 6. In the rest positions, as shown in FIG. 5, the micromirrors M are not biased by the electrostatic drive system. Two opposite polarity electrostatic fields can be selectively applied to each micromirror M, one causing it to rotate ten degrees left and the other causing it to rotate ten degrees right from the unbiased rest position. By definition herein, the left position will be referred to as the ON position and the right position will be referred to as the OFF position.

In operation with bias applied, the data for determining whether a micromirror M is in its ON position or OFF position is stored in the addressable cells 141, which preferably are SRAM memory cells. Each addressable cell 141 uniquely identifies the micromirror located immediately above it. It will be appreciated from the previous discussion of the entire apparatus 10 of FIG. 1 that the DMD controller 46 determines the positions of the individual micromirrors M. This is readily accomplished by copying the image data stored in the memory 66 into the array of addressable cells 141, each cell assuming an ON state or an OFF state so that each micromirror M assumes a corresponding ON position or OFF position.

Referring to FIG. 7, a preferred embodiment of the mirror chamber 22, which forms part of the projector 12 of FIG. 1, will now be described. The mirror chamber 22 has chamber walls 151 (shown cross-hatched) that define optical ports 153, 155, 157 and 159. The walls 151 are preferably black anodized aluminum to absorb stray light reflections. Within the mirror chamber 22 are a half-mirror 161 and a dichroic mirror 163, oriented at 45° angles as shown. The secondary laser beam 40 from the DMD module 38 enters the mirror chamber 22 through port 153 and is reflected by dichroic mirror 163 to define tailored laser beam 30, which passes out of the chamber 22 through port 155 and is ultimately directed at the patient's eye by the fundus camera 24 (see FIG. 1). Dichroic mirror 163 reflects substantially all of the light within a narrow band of wavelengths that includes the wavelength of the secondary laser beam 40. Preferably, dichroic mirror 163 operates as a half-mirror outside of its narrow band of reflection. The illuminated image of the patient's eye partially passes through mirrors 163 and 161 to exit through port 159 as image 171, which is seen by the physician through the binocular viewer 18 (see FIG. 1). A portion of the illuminated image of the patient's eye is reflected by half-mirror 161 as illuminated image 48, which is directed at the CCD camera 42 (see FIG. 1) through port 157.

An additional feature that may be included in the above-described system is the use of an aiming laser beam that is also generated by the laser source 34 and is coaxial with the primary laser beam 36. So configured, the laser source 34 will emit two different laser beams at different times during the procedure: a very low power aiming beam and a higher power primary or “therapeutic” laser beam. The aiming laser beam is selected to have an operating wavelength that is outside the narrow reflective band of the dichroic mirror 163. Thus, a portion of the aiming laser beam will be reflected downward and will illuminate a spot on the retina of the patient's eye, and a portion will pass through the mirror 163 (since it acts as a half-mirror at the wavelength of the aiming laser beam) to be absorbed by a black interior surface of the chamber 22. The illuminated spot will reflect light back up and partially through mirrors 163 and 161 to the physician's eyes, and some of the light reflected by the spot will also be reflected out through port 157 at the CCD camera.

Equipped in this way, the apparatus 10 allows the treating physician to position the projector 12 to direct the aiming laser beam at the target area of the patient's retina. The aiming beam typically will cover an area that is larger than the target area. This relationship is depicted in FIG. 1 by the circular region 95 surrounding the target area image 64 within the image of the retina on the computer screen 54. The circular area 95 represents the portion of the retina illuminated by the aiming laser beam. When the higher power primary laser beam 36 is emitted after mapping the target area image 64, the coaxial alignment with the aiming laser beam assures precise alignment of the tailored laser beam 30 with the actual target area of the patient's retina.

As previously mentioned, the invention contemplates as an option the advantageous use of a programmed system for semi-automatically defining the target area designated for irradiation. In particular, with reference again to FIG. 1, after the image of the object that is the subject of the irradiation, which may be the retina of a patient's eye, has been reproduced as the image on the computer screen 54, the computer selects a possible target area 64. For example, a lesion on the retina will appear darker than the surrounding area. The treating physician accepts the image selected by the computer 44 in a suitable way, such as by touching the screen 54 with the tip of the light pen 60 within the identified boundary of the target area 64.

Preferably, the entire image of the object on the screen 54 is digitized according to intensity to produce a gray-scale map. Software operated by the computer 44 selects one or more possible targets according to light intensity variations indicated in the gray-scale map, localized darker intensity regions indicating the presence of a lesion. A boundary line is created on the screen 54 around each such darker intensity region. The treating physician can adjust the boundary to make it larger or smaller by selecting the particular gray-scale intensity level that the computer 44 uses to outline the target area (or multiple target areas) on the screen 54. The particular gray-scale intensity level can be input to the computer 44 in various ways, such as by using the light pen 60 or a conventional mouse (not shown) to select from a table on the screen 54, or by simply typing in a character or number using the keyboard 58. Once a target area 64 is selected and its boundary defined in this manner, the process proceeds as previously described to set the states of the micromirrors in the DMD module 38 and signal the laser source 34 to fire the primary laser beam 36. Once the gray-scale map of the target lesion has been established and the outer boundary of the target area defined, the gray-scale values and shape of the target area preferably remain fixed during irradiation.

As a further refinement of the semi-automatic target mapping technique just described, the shaped laser beam 30 can be modulated in pulses to treat different regions within the target area with different doses of irradiation. This technique can be useful in different applications including the irradiation of a targeted retinal lesion. For example, if one wished to irradiate a central portion of the lesion more heavily than a peripheral portion, two boundaries can be selected, one surrounding the other. An inner boundary can be selected according to a gray-scale value that identifies the darkest center area of the lesion, and an outer boundary can be selected according to a gray-scale value that identifies the outer limits of the lesion, each selection being made with the treating physician's guidance.

Having inner and outer areas of the target area mapped in this way enables the computer system to direct the DMD module 38 to assume two different sets of ON and OFF states in which a first set of micromirrors that are within the mapped inner boundary remain ON longer than a second set of micromirrors that are within the mapped outer boundary but outside the inner boundary. Minimum and maximum pulse durations can be selected, such as 10 milliseconds and 100 milliseconds. As an example, the first set of micromirrors can be pulsed ON for 80 milliseconds of every 100 milliseconds, and the second set of micromirrors can be pulsed ON for 20 milliseconds of every 100 milliseconds. In such case, the central portion of the target lesion will receive an irradiation dose four times greater than the peripheral portion.

It will be appreciated that many variations of the foregoing laser modulation technique can be designed into the system, all such variations being within the scope of the invention. For example, a graded dose of irradiation can be applied across the lesion under treatment by using a gray-scale map of the lesion to set a pulse duration within minimum and maximum limits for each micromirror of the array within the target area identified for the lesion. Such time-division control of micromirror modulation based on gray-scale values is just one example of various alternatives for real-time laser intensity variation that can be practiced using the apparatus of the present invention.

Although a preferred embodiment of the invention has been described herein with reference to the accompanying drawings, it will be appreciated that various alternatives and modifications thereof are within the spirit and scope of the invention as set forth in the appended claims.

What is claimed is:

1. An apparatus for mapping a target area of tissue for irradiation by a laser beam without irradiating tissue surrounding the target area, comprising:
a laser source for providing a primary laser beam;
a projector having an optical system for projecting a portion of the primary laser beam onto the target area, the portion of the laser beam being tailored to conform to the shape of the target area, the projector including a light source for illuminating the target area and surrounding area to create an illuminated image;
a digital micromirror device positioned between the laser source and the projector to selectively emit a secondary laser beam directed at the projector, the digital micromirror device including an array of micromirrors, each micromirror being controllably rotatable between ON and OFF positions, the ON-position micromirrors reflecting increments of the primary laser beam that collectively define the secondary laser beam; and
a computer system for converting the illuminated image of the target area and surrounding area into a digitally stored image, displaying the stored image, responding to operator commands to map the target area within the displayed stored image, directing the digital micromirror device to assume a state that corresponds to the mapped target area, and sending a signal to the laser source to enable the laser source to direct the primary laser beam at the digital micromirror device.

2. The apparatus of claim 1, wherein the computer system comprises:
a computer having a display screen, operator controls, an input bus for receiving digital image data, and an output bus for transmitting output data and control signals;
a digital camera for converting the illuminated image into the digitally stored image and transmitting the digitally stored image to the computer over the input bus; and
a DMD controller receiving data and control signals from the computer's output bus to control the state of the digital micromirror device, the DMD controller having a memory for storing data identifying the mapped target area, a microprocessor for controlling the memory in response to the data and control signals transmitted by the computer, and a transfer bus for transmitting data and control signals to the digital micromirror device so that it will assume a state corresponding to the mapped target area.

3. The apparatus of claim 2 wherein the projector includes a mirror chamber for redirecting the secondary laser beam as the tailored laser beam directed at the target area, and for redirecting light reflected from the illuminated image toward the digital camera.

4. An apparatus for mapping a target area of an object for irradiation by a laser beam, comprising:
a laser source for providing a laser beam;
a projector for projecting at least a portion of the laser beam onto the target area of the object, the projector including:
a frame;
an imaging end supported by the frame at one end of the projector;
a viewing end supported by the frame opposite from the imaging end;
projector controls mounted on the frame for positioning the imaging end of the projector relative to the object, and for controlling the projection of the laser beam at the object; and
an optical system supported by the frame for transmitting an image of the object to the viewing end and directing the laser beam at the target area of the object;
the apparatus further comprising:
a digital camera optically coupled to the projector for creating digital data representative of an image of the object conveyed from the imaging end through the projector to the digital camera;
a computer, the computer including:
an input bus coupled to the digital camera;
an output bus;
a screen for displaying images;
a processor for receiving data from the input bus, formatting the data for display by the screen, and transmitting data onto the output bus;
a keyboard; and
an interface device in communication with the processor and cooperating with the screen for defining a map of the target area of the object within an image of the object as displayed on the screen;
the apparatus further comprising:
a DMD module optically positioned between the laser source and the projector to selectively transmit a portion of the laser beam from the laser source to the projector; and
a DMD controller in communication with the DMD module through a control bus, the DMD controller being connected to the computer's output bus for receiving data from the processor representing the map of the target area of the object created by an operator using the interface device;
whereby the operator uses the interface device to define the target area of the object within the image on the computer's screen, and uses the projector controls to direct the portion of the laser beam selected by the DMD module at the target area of the object.

5. The apparatus of claim 4 wherein the DMD module comprises:
a housing having walls defining an entry port and an exit port;
a digital micromirror device mounted in the housing; and
an optical instrument mounted in the housing for receiving the laser beam from the laser source through the entry port, the optical instrument being positioned so that the laser beam impinges on the digital micromirror device.

6. The apparatus of claim 5 wherein the digital micromirror device comprises:
a substrate; and
a plurality of micromirrors arranged in a two-dimensional array above the substrate, each micromirror being pivotally supported on a base, each base being supported by the substrate;
wherein the substrate includes addressable cells, each cell corresponding to and being disposed beneath a respective micromirror, each cell having an ON state and an OFF state, each micromirror responding to the state of its respective cell to rotate to a corresponding ON position or OFF position;
whereby the micromirrors that are disposed in the ON positions collectively transmit a portion of the laser beam from the laser source to the projector.

7. The apparatus of claim 4, wherein the projector's optical system comprises:
a mirror chamber supported by the frame;
a first lens system located between the mirror chamber and the viewing end; and
a second lens system located between the mirror chamber and the imaging end.

8. The apparatus of claim 7, wherein the mirror chamber comprises:
a first mirror for redirecting light from the DMD module toward the target area of the object; and
a second mirror for redirecting light reflected from the object toward the CCD camera.

9. The apparatus of claim 7, wherein the first lens system comprises a binocular viewer.

10. The apparatus of claim 9, further comprising a filter box located between the mirror chamber and the binocular view.

11. The apparatus of claim 7 for use in ophthalmic surgery wherein the second lens system comprises a fundus camera.

12. The apparatus of claim 4, wherein the interface device comprises a light pen, whereby the operator uses the light pen to manually draw a boundary around the target area within the image of the object displayed on the screen.

13. A method of selective irradiation of an object comprising:
creating a digital image of the object;
mapping a target area on the image;
transmitting data corresponding to the mapped target area to a digital micromirror device to cause the digital micromirror device to assume a state for selectively reflecting light along first path and a second path;
directing laser light at the digital micromirror device when it is in its selectively reflecting state;
absorbing the laser light reflected along the second path; and
redirecting the laser light reflected along the first path to impinge upon the target area of the object.

14. The method of claim 13 wherein the mapping step is performed by creating a visual image of the digital image on a computer screen, and then manually drawing a boundary around the target area using an interface device.

15. The method of claim 14 wherein the manually drawing step is performed using a light pen as the interface device.

16. The method of claim 13 further comprising:
tracking movement of the target area as part of the mapping step;
repeating the transmitting step to continuously cause the digital micromirror device to update its selectively reflecting state to change the content of the light reflected along the first path to correspond to the movement of the target area.

17. The method of claim 16 further comprising:
inhibiting emission of laser light to prevent laser light from impinging on the target area whenever the mapped target area of the digital image moves more than a predetermined amount.

18. The method of claim 17 wherein the predetermined amount is determined by the limits of the digital image within which the mapped target area can move and be fully exposed to irradiation by the laser light.

19. The method of claim 13 wherein the mapping step is performed by creating a gray-scale map of the digital image, and choosing a gray-scale value that defines a boundary around a target area based upon established criteria for differentiating the target area from its surrounding area.

20. The method of claim 19 wherein the gray-scale map within the target area is used to modulate the digital micromirror device to vary the irradiation dosage within the target area of the object in proportion to the varying gray-scale values therein.
